(19) United States
(12) Patent Application Publication
(10) Pub. No.: US 2010/0112014 A1
(43) Pub. Date: May 6, 2010

(54) NOVEL HYDROGEL COMPOSITIONS AND METHODS OF USING

(76) Inventors: Ryan J. Gilbert, Houghton, MI (US); Benton C. Martin, Flushing, MI (US); Matthew M. Pap, Verona, WI (US); Eric J. Minner, Houghton, MI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/421,388

(22) Filed: Apr. 9, 2009

Related U.S. Application Data
(60) Provisional application No. 61/044,217, filed on Apr. 11, 2008.

(51) Int. Cl. A61K 31/717; A61K 9/00; A61P 25/00; A61K 35/00; A61K 38/00; A61K 38/29; A61K 38/18; A61K 38/39

(52) U.S. Cl. 424/400; 514/57; 424/93.1; 514/2; 424/93.7; 424/93.72; 514/12

(57) ABSTRACT

Disclosed are novel agarose, methylcellulose, and dextran hydrogels. Further disclosed are methods of making and using the hydrogels.

### CROSS-REFERENCE TO RELATED APPLICATIONS

This application claims priority to U.S. Provisional Patent Application No. 61/044,217, filed Apr. 11, 2008, and incorporated herein by reference in its entirety.

### BACKGROUND

Injury, whether traumatic or degenerative, to the nervous system or other areas of the body, is a debilitating problem for thousands of people worldwide. Additional solutions for the regeneration of tissue are needed.

### BRIEF SUMMARY OF THE INVENTION

In one aspect, the present invention provides a hydrogel comprising methylcellulose, agarose and dextran. In another aspect, the present invention also provides a composition comprising the hydrogel and a scaffold.

In a further aspect, the present invention provides a method of making the hydrogel comprising combining a suspension of agarose and methylcellulose and dextran solution; and mixing the suspension and solution at low temperature.

In another aspect, the present invention provides, a method of treating nervous system injury comprising administering the hydrogel to a subject in need thereof. In yet another aspect, the present invention provides, a method of regenerating tissue comprising contacting damaged tissue with the hydrogel. In another aspect, the present invention provides a method of enhancing tissue regeneration comprising administering the hydrogel to a subject in need thereof. The tissue is suitably nerve tissue, connective tissue, muscle tissue or skin.

In a further aspect, the present invention provides a method of treating a subject having an artificial joint comprising administering the hydrogel to the artificial joint site. In yet another aspect, the present invention provides a method of facilitating nerve growth comprising contacting a region in need of nerve growth with a hydrogel.

### BRIEF DESCRIPTION OF THE DRAWINGS

FIG. 1 is a schematic diagram of a syringe pump, force measurement system, and syringe set-up to quantify the ease of injectability of the hydrogel compositions of the invention.

FIG. 2A is a graph of gelation temperatures of three agaroses when not mixed with methylcellulose. FIG. 2B is a graph of temperatures at which mixtures of agarose and methylcellulose solidified.

FIG. 3A is a graph of the time required for mixtures of agarose and methylcellulose to solidify in a 37° C. water bath. FIG. 3B and FIG. 3C are modulus curves of the four hydrogels.

FIG. 4A are SEM images of agarose and methylcellulose mixtures. FIG. 4B is a graph of the average pore size of hydrogel analyzed from SEM images. FIG. 4C is a graph of the pore size distribution of pores imaged within each hydrogel species.

FIG. 5 is a graph of the percent hydrogel dissolved on various days.

FIG. 6A are images of live and dead cells with agarose and methylcellulose mixtures. FIG. 6B is a graph of the average ratio of live cells to total cells from hydrogel blends and methylcellulose.

FIG. 7 are images of cells in the presence of hydrogel blends.

FIG. 8 is a graph of gelation times of hydrogels containing incremental amounts of neutral dextran.

FIG. 9 is a graph of gelation times of hydrogels with dextran and incremental volumes of methacrylic acid (MAA).

FIG. 10 is a graph of gelation times of hydrogels containing incremental amounts of dextran and 60 mM MAA.

FIG. 11 is a graph of gelation temperatures of hydrogels.

FIG. 12 are SEM images and average pore size of hydrogels.

FIG. 13 is a graph of elastic modulus versus time for various hydrogel compositions.

FIG. 14 are images of dissociated dorsal root ganglia cells cultured in the presence of agarose/methylcellulose hydrogel with and without dextran.

### DETAILED DESCRIPTION

Among other things, the present invention provides novel hydrogel compositions, method of making the hydrogel compositions, and methods of using the hydrogel compositions to treat various conditions. The hydrogels of the present invention comprise agarose, methylcellulose and dextran. In some embodiments, the hydrogels comprise about 3% to about 20% (w/v) methylcellulose, about 10% to about 12% (w/v) methylcellulose, about 5% to about 9% (w/v) methylcellulose, or about 7% (w/v) methylcellulose. Suitable methylcelluloses include, but are not limited to, Methocel A15 Premium LV (Dow Chemical Company, Midland, Mich.). As used herein, the weight/volume percentages may describe the percentages used to make the hydrogel.

The hydrogels of the present invention may comprise about 0.5% to about 5% (w/v) agarose or about 0.75% (w/v) or about 1.5% (w/v) agarose. Various agaroses are commercially available that have differing degrees of methoxyl substitution. Suitable agaroses include, but are not limited to, Seaprep® agarose (Lonza, Basel, Switzerland), Metaphor® (Cambrex Bio Science Rockland Inc., Rockland, Me. or Lonza, Basel, Switzerland), and NuSieve 3:1® (Lonza, Basel, Switzerland).

In some embodiments, the hydrogels comprise about 20 to about 500 mg dextran per about 15 mL of hydrogel, and suitably about 100 mg dextran per about 15 mL of hydrogel. The average molecular weight of the dextran may be about 10 to about 500 kDa molecular weight, or about 20 to 250 kDa molecular weight, suitably about 150 kDa molecular weight. Suitably, the dextran may be at least partially positively charged prior to addition to the hydrogel. In some embodiments, the dextran is substantially completely positively charged. Dextran can be positively charged by treating it with an acid, such as methacrylic acid. Other suitable acids would be known to one of ordinary skill in the art.

In some embodiments, the hydrogels contain at least one additional bioactive component. It is envisioned that almost any suitable bioactive component may be added to the hydrogels. As used herein, “bioactive” is intended to indicate the ability to facilitate a tissue or cellular response. Those additional bioactive components include, but are not limited to, cells, peptides, polypeptides, polymers, and small molecules, and combinations thereof. Suitably, the cells include, but are not limited to, platelets, Schwann cells, neurons, keratinocytes, fibroblasts, chondrocytes, bone formation cells (such as osteoblasts and osteoclasts), myocytes, and stem cells. Peptides or polypeptides useful in the present invention include, but are not limited to, cytokines (such as interleukins), glutathione, parathyroid hormone, growth factors (such as fibroblast growth factors, nerve growth factors, insulin-like growth factors, platelet-derived growth factors, vascular endothelial growth factors, and bone morphogenic proteins), neurotrophic factors (such as NT-3), coagulation agents, laminin, fibronectin, collagen, and proteoglycans. Polymers include, but are not limited to, polyethylene glycol. Small molecules suitable for use in the present invention include, but are not limited to, antibiotics, anti-fungals, coagulation agents, and immunosuppressants. Temperature-sensitive bioactive components are suitable for use in the present invention. The hydrogels of the present invention may be made by a chilled fabrication step that allows for the incorporation of temperature-sensitive bioactive components without affecting the activity of the temperature-sensitive bioactive components.

In some embodiments, the hydrogels of the present invention contain at least one neuroinductive agent, such as nerve growth factor, neurotrophic factors, (such as NT-3), and laminin. In other embodiments, the hydrogels of the present invention contain at least one osetoinductive agent, such as bone morphogenic proteins, platelet rich plasma, synthetic peptides, and parathyroid hormone

When present in the hydrogel composition, the at least one additional bioactive component generally is present at a volume concentration of about 0.01 percent to about 90 percent based on the total volume of the hydrogel composition.

Suitably, the hydrogels of the present invention solidify naturally without the need for ultraviolet light or chemical cross linkers. In some embodiments, the hydrogels solidify at physiological temperature in about 3 to 4 minutes. Alternatively, the hydrogels solidify at physiological temperature in less than about 10 minutes or less than about 15 minutes. In other embodiments, the hydrogels solidify at physiological temperature in no more than about 10 minutes or no more than about 15 minutes.

In some embodiments, the hydrogels of the present invention have a pore size (diameter) of from about 10 microns to about 100 microns or about 20 to about 80 microns or about 20 to 50 microns. The pore size may affect release of the additional bioactive components, such as therapeutic agents, which are incorporated in the hydrogel. For example, larger pore size may result in faster release of the additional components and smaller pore size may result in slower release of the additional components.

The present invention also provides a composition comprising the hydrogel of the present invention and a scaffold. Suitable scaffolds include, but are not limited to, nerve guidance channels, bone constructs, metal cages and ceramic frameworks. In certain embodiments, the hydrogel is at least partially contained within the scaffold. In one embodiment, the scaffold may comprise calcium phosphate.

In some embodiments, the nerve guidance channels comprise a set of aligned polymer fibers. Suitable polymer fibers include, but are not limited to, poly-L-glycolic acid, poly-L-lactic acid, blends of poly-L-glycolic acid and poly-L-lactic acid, collagen, laminin, fibrin and combinations thereof.

The present invention also provides a method of making the hydrogels. In one embodiment, agarose and methylcellulose may be combined with a first solvent to form a suspension. As used herein, “suspension” includes a mixture wherein at least one of the components is at least partially dissolved. One of ordinary skill in the art would be able to determine a suitable solvent, for example, phosphate buffered saline (PBS). The first solvent may be heated to about 70° C. to about 100° C., suitably about 80° C., prior to or after the addition of agarose and methylcellulose. A separate solution of dextran in a second solvent is formed. The second solvent may be the same as or different from the first solvent. Suitably, the first and/or the second solvent is not an organic solvent. The dextran solution and the suspension are combined, and the entire mixture is mixed to form a hydrogel. In some embodiments, the dextran solution may be at a first low temperature prior to combination with the suspension. Suitably, the mixture is mixed at second low temperature. The first and second low temperatures may be the same or different. In some embodiments, the first and/or second low temperatures are less than about 10° C. or less than about 5° C. or less than about 0° C. or less than about −10° C.

In another embodiment, dextran may be added to the solution with the agarose and methylcellulose. As used herein, “added” includes added at the same time, added at about the same time, or added sequentially.

The hydrogel may be centrifuged and stored in a refrigerator. Alternatively, the hydrogel may be freeze-dried or dehydrated for storage or transport.

Acids, such as methacrylic acid, and bases, such as NaOH, may be incorporated with the agarose, methycellulose, and/or dextran to achieve a desired pH. Such a desired pH may be a physiological pH. For example, the final pH of the hydrogel may be about 7.3 to about 7.5.

When present, the additional bioactive component(s) can be added to the hydrogel with the dextran.

The present invention also provides methods of using the hydrogels. For example, the hydrogels may be used to treat injuries, whether traumatic or degenerative, to the nervous system (either central or peripheral), to connective tissue, such as bone, tendons, ligaments and cartilage, to muscle tissue and to skin.

In one embodiment, the present invention provides a method of regenerating tissue comprising contacting damaged tissue with a hydrogel. In another embodiment, the present invention provides a method of enhancing tissue regeneration comprising administering a hydrogel to a subject in need thereof. Tissues that may be regenerated using the hydrogels of the present invention include nerve tissue, connective tissue, muscle tissue and skin.

In another embodiment, the invention provides a method of facilitating nerve growth comprising providing a hydrogel to a region in need of nerve growth. In further embodiment, the invention provides a method of treating nervous system injury comprising administering a hydrogel to a subject in need of treatment. In some embodiments, the nervous system is the central nervous system. One form of central nervous system injury that may be treated with the hydrogels of the present invention is spinal cord injury. In other embodiments, the nervous system is the peripheral nervous system. One example of peripheral nervous system injury is diabetic neuropathy.

In yet another embodiment, the present invention provides a method of treating connective tissue injury comprising administering a hydrogel to a subject in need of treatment. Connective tissue injuries that may be treated with the hydrogels of the present invention include, but are not limited to, broken bones, long segmental diasphyseal bone loss, bone loss resulting from osteomyelitis or osteonecrosis or osteosarcoma or osteoporosis, torn ligaments (e.g. anterior cruciate ligament, posterior cruciate ligament, lateral collateral ligament, or medial collateral ligament) and torn tendons (e.g. patellar tendon). In another embodiment, the present invention provides a method of treating skin injuries comprising administering a hydrogel to a subject in need of treatment. In a further embodiment, the present invention provides a method of treating muscle injuries comprising administering a hydrogel to a subject in need of treatment. The present invention also provides a method for treating a subject having an artificial joint. In particular, the hydrogel may be administered around the site of the artificial joint, either during placement of the artificial joint or post-surgery, to facilitate integration of the artificial joint into the surrounding tissue.

As used herein, “treating” encompasses situations where the hydrogel and/or the additional bioactive component supply the intended response. As used herein, “contacting with a hydrogel” or “administering a hydrogel” include contacting or administering with a hydrogel according to the present invention or a composition comprising a hydrogel according to the present invention. It is desirable that upon administration or contact, the hydrogel conforms to the geometry of the injury site. The hydrogel may be delivered to the desired site via injection. In some embodiments, the hydrogel is administered at refrigerated temperature.

Suitably, a therapeutic amount of the hydrogel or hydrogel composition is administered to a subject. The subject can be any animal, including mammals, such as dogs, cats and humans. The term “therapeutic amount” refers to the amount required to promote tissue repair or regeneration as evidenced by growth of new tissue in an area previously lacking such tissue. The therapeutic amount will primarily be determined by the size and type of injury being treated. Typically, the volume of hydrogel applied is about 1 to 60 mL, but could be greater, especially in the case of large injuries. Suitably, the therapeutic amount is sufficient to provide uniform scaffolding for cellular attachment and regeneration in the area where tissue regeneration is needed.

### EXAMPLES

The following examples are intended to illustrate the invention to those skilled in the art and should not be interpreted as limiting the scope of the invention set forth in the claims.

### Example 1

### Creation of Agarose/Methylcellulose Blends

Three agaroses were chosen to be tested for combination with methylcellulose. A type of methylcellulose called METHOCEL* A15 Premium LV (Dow Chemical Company, Midland, Mich.) was used because of its successful previous use within the central nervous system (Gupta, D. et al. Biomaterials 2006, 27, 2370-2379).

5%, 7%, and 9% (wt/vol) methylcellulose hydrogel blends were prepared and tested for gelation temperature. It was observed that 5% methylcellulose required temperatures higher than physiological temperatures to solidify while 9% methylcellulose had high viscosity making it difficult to experiment with and causing the hydrogel to solidify at temperatures much lower than the optimum 37° C. 7% methylcellulose solidified at approximately 37° C. and was chosen to be the concentration used in combination with agarose.

The concentrations of agarose to be used were chosen based on gelation tests with mixtures containing 7% methylcellulose and varying amounts of agarose. For each type of agarose, the concentrations of that hydrogel that exhibited the fastest gelation time and a gelation temperature of approximately 37° C. were chosen to be tested further. This resulted in 1.5% (wt/vol) Metaphor® (Cambrex BioSciences Rockland Inc., Rockland, Me.) and SeaPrep® (Lonza, Basel, Switzerland) blends, using 225 mg of agarose powder and 0.75% (wt/vol) NuSieve 3:1® (Lonza, Basel, Switzerland) requiring 112.5 mg of agarose powder.

To create each blend, the desired masses were first measured. The agarose was then deposited into a beaker and mixed with 7.5 ml of 7.4 pH sterile phosphate buffer saline (PBS). The beaker was covered with aluminum foil and placed on a hot plate with constant stirring until the mixture was clear and colorless. Aluminum foil was removed and the volume was re-measured to account for evaporation. If evaporation occurred, sterile water was added so that the total volume was 7.5 ml. The hot plate and beaker were moved to the sterile hood. While covered with aluminum foil, the mixture was heated before being filtered into a sterile beaker using a Millex 0.22 micron filter (Millipore, Bedford, Mass.). Methylcellulose powder was sterilized using ethylene oxide gas. The methylcellulose addition utilized a previously published method for mixing methylcellulose (Tate, M. et al. Biomaterials 2001, 22, 1113-1123). The agarose hydrogel solution was heated in the sterile hood until it reached over 80° C. When the PBS is above this temperature, the methylcellulose cannot dissolve and can easily be dispersed throughout the liquid (The Dow Chemical Company, Methocel Cellulose Ethers Technical Handbook September 2002, 1-30). While still on the hot plate, 1050 mg (7%) of methylcellulose was added to the sterilized 7.5 ml agarose solution. The mixture was then stirred until all the particles were dispersed. When a cloudy uniform liquid had formed, the beaker was removed and 7.5 ml of sterile PBS was added directly after being removed from a 4° C. environment. The beaker was placed on ice and the mixture was stirred for 20 minutes before being transferred to a sterile 15 ml conical tube. The tubes were then centrifuged to remove air bubbles that form during stirring.

Statistical Analyses. For all examples, ANOVA was run to determine statistical significance between the temperatures when agarose gelled, the time it took the hydrogels to blend, and the percent of hydrogel that had degraded. The Tukey-Kramer HSD test of all pairs was used to determine statistical significance between hydrogels in temperature, time, pore size characterization, and degradation studies. These calculations were performed using JMP IN 5.1 software with a significance level of α=0.05 (SAS Institute Inc., Cary, N.C.). All data are reported as the mean plus or minus the standard deviation.

### Example 2

### Gelation Temperature and Time Tests of Agarose/Methylcellulose Blends

Two important factors in determining the capacity of hydrogels to be used in vivo are their ability to solidify at the physiological temperature of 37° C. and the duration of time required for each blend to solidify at a constant temperature of 37° C. Gelation temperature was measured using the inverted test tube test (Gupta, D. et al. Biomaterials 2006, 27, 2370-2379). The three agarose solutions (1.5% Metaphor®, 0.75% NuSieve 3:1®, and 1.5% SeaPrep®) were melted and then equilibrated at 37° C. for at least three hours prior to temperature testing. The hydrogels were submerged in a 50° C. water bath for one minute, and then the temperature of the bath was lowered 1° C. per minute until gelation occurred. When the mixture no longer flowed while inverted, it was considered solidified, and the temperature was recorded.

Agarose and methylcellulose have inverse thermal gelling properties. Consequently, the blends solidify at higher temperatures than do the agarose hydrogels alone (Aymard, P. et al. Biopolymers 2001, 59, 131-144). The blends started at 15° C. in the water bath where they equilibrated for five minutes, and the water temperature was increased 1° C. per minute until gelation occurred.

To begin the gelation time test, 1 ml liquid hydrogel was pipetted into a 1.5 ml micro-centrifuge tube. The tube was allowed to equilibrate at room temperature for 20-30 minutes. It was then placed in a 37° C. water bath and checked every minute by a blinded observer to see if the liquid solidified. Time was stopped when the hydrogels were outside of the water and was restarted when they were put back into the water. Gelation point was determined by the inverted test tube method. Hydrogels were inverted and firmly shaken twice to be sure that there was no adhesion to the side of the microcentrifuge tube before declaring each a solid. The time that it took for the liquid to change to a solid was recorded, and this value was denoted as the gelation time.

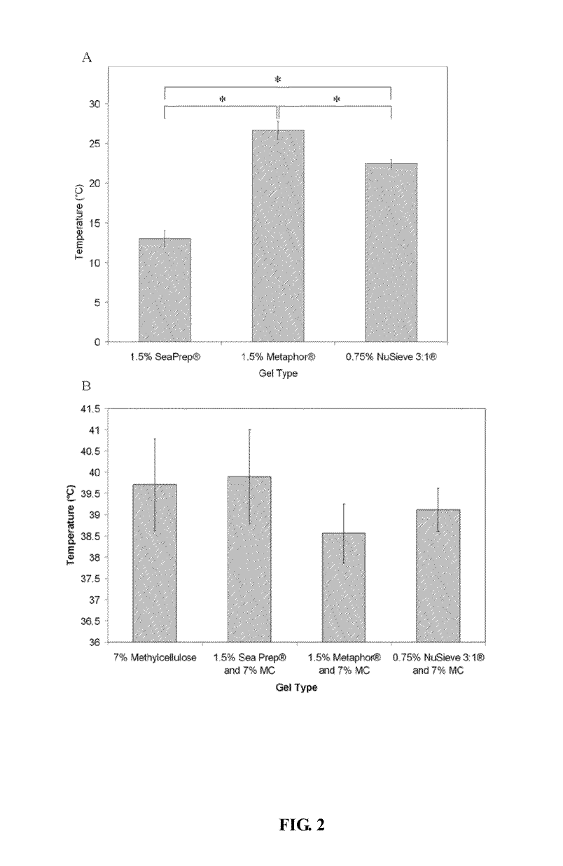

Gelation Temperature Results. The gelation temperatures of the plain agarose hydrogels were lower than physiological temperatures. 1.5% SeaPrep® proved to have the lowest gelation temperature, 0.75% NuSieve 3:1® had the next highest, and 1.5% Metaphor® had the highest gelation temperature. The plain agaroses all had significantly different gelation temperatures when compared to each other (FIG. 2A).

The gelation temperature tests showed similar results for all of the hydrogel blends and methylcellulose. On average the 1.5% SeaPrep® agarose and 7% methylcellulose blend produced a hydrogel with the highest gelation point. The 0.75% NuSieve 3:1® and 7% methylcellulose blend solidified at the lowest average temperature. However, there was no statistical difference in the gelation temperature between different experimental groups. The agarose percentages chosen (1.5% SeaPrep®, 1.5% MetaPhor® and 0.75% NuSieve®) when mixed with 7% methylcellulose all gelled at approximately 37° C. when subjected to the temperature test. The results of this test are shown in FIG. 2B, with each agarose mixture hydrogel compared to 7% methylcellulose hydrogel. (*) denotes statistical significance when comparing the plain agarose hydrogels to each other using the Tukey-Kramer HSD test.

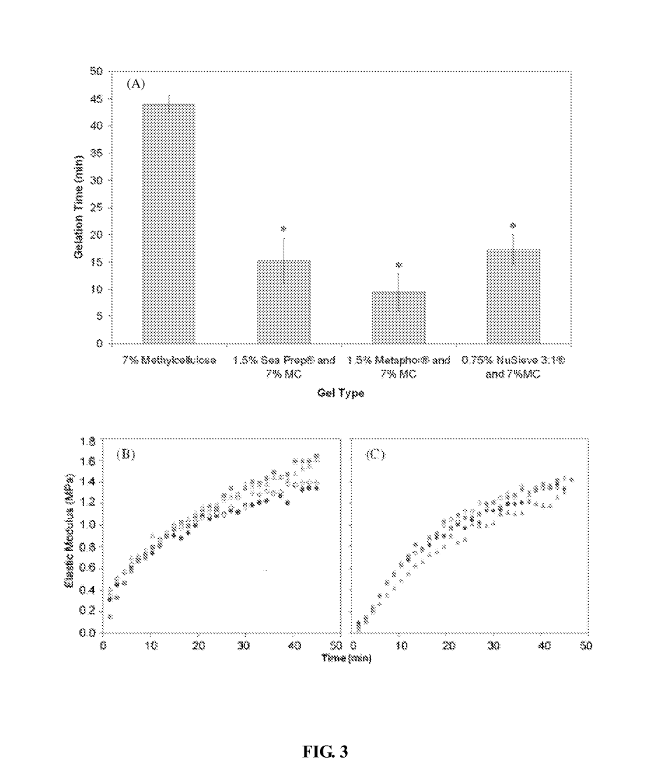

Gelation Time Results. Significant differences were seen in the time required for methylcellulose to solidify versus the gelation time of the three blended hydrogels as shown in FIG. 3. In FIG. 3, (*) denotes statistical significance when comparing the plain agarose hydrogels to each other using the Tukey-Kramer HSD test. The hydrogel that solidified fastest was the 1.5% Metaphor® with 7% methylcellulose which solidified on average after 9.5 minutes. The longest gelation time for any of the blended hydrogels was 0.75% NuSieve 3:1® with 7% methylcellulose which took an average of 17.3 minutes to solidify. 1.5% SeaPrep® with 7% methycellulose took an average of 15.2 min to solidify. These were significantly lower than the 7% methylcellulose which averaged 44 minutes to solidify at 37° C.

### Example 3

### Rheometry of Agarose/Methylcellulose Blends

Small amplitude oscillatory rheometry was conducted using a Bohlin C-VOR Rheometer (Malvern Instruments Ltd, Malvern, Worcestershire, UK) and provided quantified values for gelation time. Frequency sweeps from 10 to 100 radians were repeated isothermically at both 30° C. and 37° C. for 45 min. Each of the hydrogel samples, in triplicate (n=3), were heated using a convection oven, utilizing heated nitrogen air to maintain the temperature during the test.

The rheological testing showed that the elastic modulus of the hydrogels increased immediately once placed into a heated environment. The modulus then continued to gradually increase over the course of 15 min. All of the hydrogels had similar increasing modulus curves. FIGS. 3(B) and (C) compare the four tested hydrogels at 37° C. and 30° C., respectively, at the frequency of 100 radians. The chart plots the rising modulus of the gels over the course of thirty iterations of a frequency sweep from 10 to 100 radians lasting 1.5 min each. The modulus values at 100 radians are shown here (all frequencies showed similar results). All hydrogels blends and plain methylcellulose at 37° C. had a modulus value of 1.0 MPa after residing in the heated chamber for 15 min. Each hydrogel was tested three times. However, since there was little variability seen within a particular hydrogel group, no error bars are reported.

### Example 4

### Quantification of Force Required to Inject Hydrogel of Agarose/Methylcellulose blends

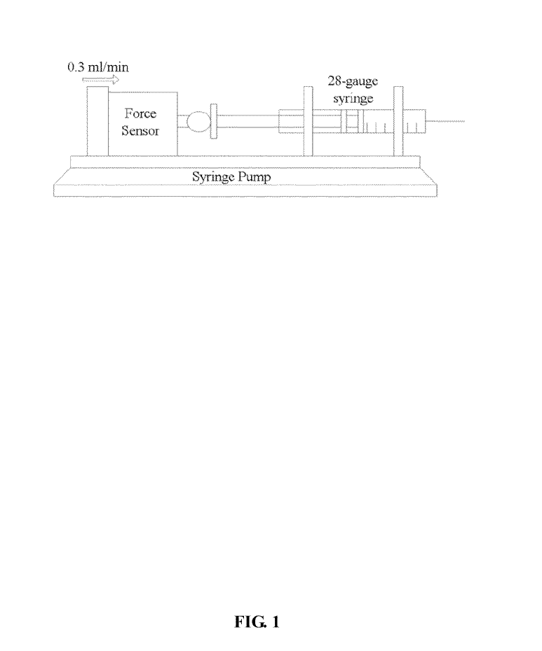

The hydrogels of this study were created to be injected into an injury site. Once the hydrogels were mixed, the hydrogels had visibly high viscosity. To quantify the injection force for each hydrogel, a force-sensing injection pump experiment was designed (FIG. 1). The injection force was measured using a NE-1600 syringe pump (New Era Pump Systems, Farmingdale, N.Y.) and a Vernier Dual Range Force Sensor (Vernier Software and Technology, Beaverton, Oreg.) in triplicate (n=3). The force measurement system was set up to measure compression. 28-gauge 100 cc micro-fine insulin needles (BDMedical, Franklin Lakes, N.J.) were filled with 20 cc of each hydrogel. The syringe was placed in the syringe pump and injected while the compressive force was being measured. The highest force recorded by the measurement system was recorded. The force was measured at injection rates of 0.1 ml min-1, 0.2 ml min-1 and 0.3 ml min-1.

7% methylcellulose proved to require less force to inject than any of the agarose and methylcellulose blends, but all hydrogels could be injected through a 28-gauge syringe. The force required steadily increased as the rate of injection increased. The force required to inject the hydrogel blends was noticeably larger than the force needed to inject methylcellulose. The hydrogels requiring the most force to inject were the Metaphor® and SeaPrep® blends. Table 1 shows the maximum forces recorded at the highest injection rate tested (0.3 ml/min).

TABLE 1
Maximum forces recorded at 0.3 ml/min injection rate

| | |
|---|---|
| 7% Methylcellulose | 15.37 ± 1.38 N |
| 1.5% SeaPrep ® and 7% Methylcellulose | 19.83 ± 2.48 N |
| 1.5% Metaphor ® and 7% Methylcellulose | 22.08 ± 2.12 N |
| 0.75% NuSieve ® and 7% Methylcellulose | 20.43 ± 1.62 N |

### Example 5

### Structural Characterization and Pore Size Evaluation of Agarose/Methylcellulose Blends

To determine whether mixing agarose with methylcellulose affected the pore size of the methylcellulose, and to evaluate the potential drug release characteristics, the pore sizes of the blends and plain methylcellulose were evaluated. Hydrogels of 7% methylcellulose blended with the three different types of agarose were characterized using scanning electron microscopy (SEM). To prepare the hydrogels for SEM, approximately 2 ml of hydrogel was pipetted into a small ring mold and incubated until the hydrogel solidified. The mold was then removed and the samples were snap-frozen using liquid nitrogen. The samples were then freeze-dried. Next, the samples were coated using a Hummer 6.2 Sputter Coater (Anatech LTD, Denver, N.C.) with a 10 nm thick layer of platinum/palladium and then analyzed with a Hitachi S4700 field emission scanning electron microscope. Four images were obtained at a magnification of 250× and four images were obtained at a magnification of 900×. The pore sizes from all eight images were measured using Adobe Photoshop and the scale recorded from the SEM. The pore size was counted as the diameter of the pore. For oblong pores, the shortest diameter was recorded because it was taken to be the limiting diameter. The eight largest pore sizes for each hydrogel were used to chart pore size and examine differences between the hydrogels.

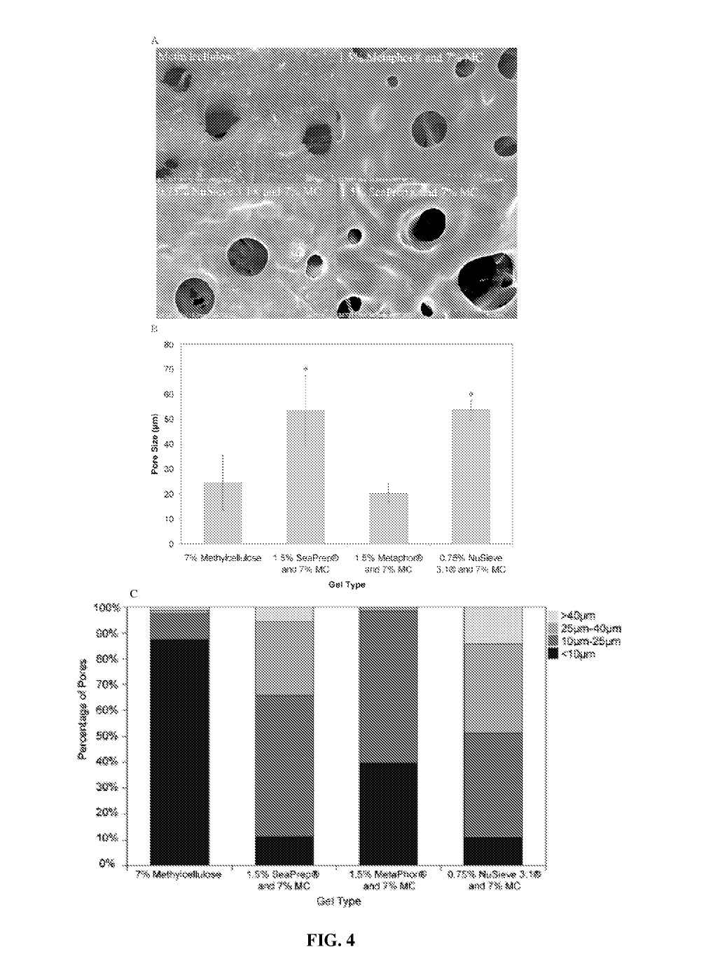

SEM images were captured of each of the hydrogels. FIG. 4A contains representative images of each of the hydrogels. FIG. 3B shows the average pore size of hdyrogels analyzed from the SEM images. Hydrogels were analyzed using the scale given on the image and the eight largest recorded were averaged together to generate the pore size value. (*) denotes statistical significance when comparing the 1.5 SeaPrep® agarose/7% methylcellulose blend and the 0.75% NuSieve® 3:1/7% methylcellulose blend to plain 7% methylcellulose using the Tukey-Kramer HSD test. Characterization of the pore sizes within the different hydrogels (FIG. 3B) showed that the 7% methylcellulose hydrogel had the highest percentage of pores less than 10 μm in diameter. The addition of agarose to the hydrogel created larger pores. Of the hydrogel blends, the 1.5% MetaPhor®/7% methylcellulose hydrogel blend had a pore size distribution most similar to that of 7% methylcellulose. The 1.5% SeaPrep®/7% methylcellulose blend and 0.75% NuSieve 3:1®/7% methylcellulose blend had more pores of larger diameter than the 1.5% MetaPhor®/7% methylcellulose blend and the methylcellulose control.

### Example 6

### In Vitro Dissolution of Agarose/Methylcellulose Blends

For nerve regeneration applications, it is important that the injected hydrogel persist in the injury cavity to allow for sustained release of therapeutics and to hold scaffolds in place. To study dissolution, a simple test (Gupta, D. et al. Biomaterials 2006, 27, 2730-2379) involving the biomaterial bathed in PBS at 37° C. is able to provide an in vitro simulation of hydrogel dissolution. The mass of eighteen 1.5 ml micro-centrifuge tubes were measured and recorded. 100 μl of the hydrogel blend solutions were injected into the bottom of the centrifuge tubes in triplicate (n=3). The solutions were allowed to solidify by incubating them for approximately one hour at 37° C. For each blend, the mass of the solution in the tubes was calculated by measuring the mass of the tubes with the hydrogel inside. Three of the tubes were freeze-dried immediately and the freeze-dried weights were recorded. 800 μl of PBS was then added into the fifteen remaining micro-centrifuge tubes on top of the solidified hydrogel mixture. At the same time every day, PBS was poured off and new PBS was added. At 1, 4, 7, 14, and 28 days, three of the tubes were removed and massed for dissolution values and to observe swelling effects. They were then freeze-dried, so that only polymer remained, and then massed for dissolution values. The percentage of dissolution was calculated using the following equation:

$$\% Dissolution ( t ) = 100 - ( \frac{{W}_{d} ( t ) / {W}_{w} ( 0 )}{{W}_{d 1} ( 0 ) / {W}_{w 1} ( 0 )} 100 )$$

where Wd1(0) is the mass of dry control hydrogel polymer, Ww1(0) is the mass of wet control hydrogel, Wd(t) is the mass of the dry polymer after dissolution, Ww(0) is the initial mass of the hydrogel before dissolution.

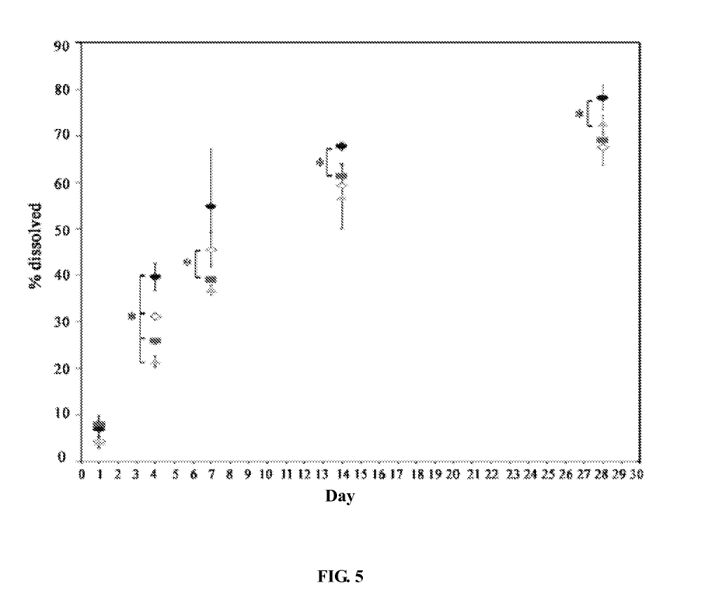

The hydrogels showed very minimal swelling in the PBS solution, holding at or below their original weight of water throughout the 28 days (data not shown). Plain 1.5% SeaPrep®, 0.75% NuSieve 3:1®, and 1.5% Metaphor® agarose all dissolved completely when left in PBS in a 37° C. incubator for only seven days, making it difficult to record percent degraded due to hydrogel instability (data not shown). As seen in FIG. 5, the three blends have markedly similar dissolution trends. In FIG. 5, the percent of hydrogel that was dissolved was measured on day 1, 4, 7, 14, and 28, for (diamonds) 1.5% SeaPrep® with 7% methylcellulose, (squares) 0.75% NuSieve® 3:1 with 7% methylcellulose, (triangles) 1.5% Metaphor® with 7% methylcellulose, and (circles) the base 7% methylcellulose. (*) denotes statistical significance when comparing the agarose/methylcellulose blends to each other and to plain methylcellulose using the Tukey-Kramer HSD test. Each hydrogel blend was approximately 70% degraded by the end of 28 days in the cycled PBS solution. Base 7% methylcellulose showed itself to be a weaker hydrogel that dissolves more rapidly than the hydrogel blends, dissolving over 75% in 28 days.

### Example 7

### Neuronal Viability with Agarose/Methylcellulose Blends

Dorsal root ganglion (DRG) cells were isolated from embryonic stage nine chick embryos using techniques approved by the Institutional Animal Care and Use Committee at Michigan Technological University. Gathered ganglia were placed into Hank's Balanced Salt Solution (HBSS) (Media Tech, Herndon, Va.), pipetted into a conical tube and centrifuged. The HBSS was removed and 1 ml of 0.25% trypsin solution added. The trypsin were incubated with the cells for 20 min, and then centrifuged and removed. 2 ml of growth medium (88% Dulbecco's Modification of Eagle's Medium (Mediatech Inc., Herndon, Va.), 10% fetal bovine serum (FBS) and 2% penicillin streptomycin) was added to the cells and a fire-blown Pasteur pipette was used to dissociate the cells. The solution was then centrifuged, the growth medium removed and 1 ml of neural growth media (neurobasal medium 1-glutamine, B-27, and penicillin/streptomycin) (Invitrogen, Carlsbad, Calif.) was added. The cells were then placed into a small culture dish and allowed to incubate for 30 min. After 30 min, the culture dish was agitated and the neural basal media consisting of neurons was removed and placed back into a conical tube. 10 μl of the cell suspension was then stained with Trypan Blue, to account for cells that may have died during the dissociation process.

Neurons were plated onto laminin coated 6-well plates (BD Biocoat, San Jose, Calif.) and allowed to attach for 48 h in a tissue culture incubator in the presence of neural growth medium supplemented with NGF (Calbiochem, San Diego, Calif.) for a final concentration of 50 ng ml¹. After 48 h, the neural growth medium was removed and approximately 50 μl of chilled hydrogel (refrigerated temperature) was placed on top of the neurons. The cells with hydrogel were then placed into a tissue culture incubator for 15 min to allow for hydrogel solidification, after which neural growth medium supplemented with NGF was again added. The cultures were then allowed to persist for 48 h and then the cultures were stained. Neural growth medium was removed from the hydrogels and the hydrogels were washed with PBS. Calcein-AM stock solution was made by dissolving 1 mg of calcein-AM (Sigma, St Louis, Mo.) into 250 μl of DMSO. 5 μl of the calcein-AM stock was added to 10 ml of PBS. The PBS/calcein-AM solution was added on top of each hydrogel. It was found that subjecting the hydrogels to a room temperature environment for an extended period of time caused the solidified hydrogels to dissolve. To prevent dissolution, the hydrogels were placed back into the incubator to allow the stains to interact with the cells. The stain was removed after 30 min and the hydrogels were again washed with PBS for 15 min. The cell cultures were analyzed using a Zeiss Axiovert 200M inverted fluorescence microscope with Apotome. The hydrogels were analyzed to confirm that the application of the hydrogel did not alter neuronal viability or morphology. Each of the hydrogels were tested three times (n=3), meaning that each hydrogel was added to cultures prepared on three separate days. Four images in different locations of the hydrogel were also captured. The hydrogels were analyzed by counting dead and live cells within each of the four images, then calculating the ratio of live cells to dead cells. Two separate blinded observers counted the number of live and dead cells within each captured image, and the results were averaged.

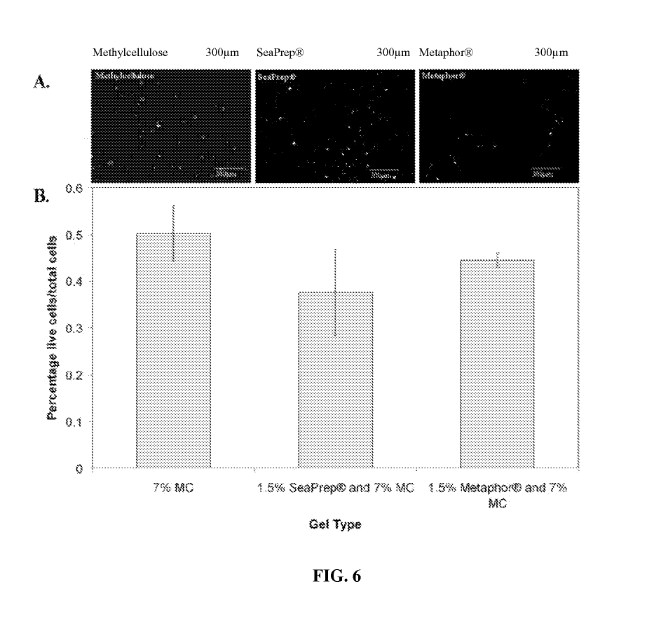

FIG. 6A show images taken with a Zeiss Axiovert 200M inverted fluorescence microscope with Apotome showing live and dead cells stained with calcein and ethidium homodimer cultured in base methylcellulose, 1. % Metaphor® agarose and 7% methylcellulose blend, and 1.5% SeaPrep® agarose and 7% methylcellulose blend. As seen in FIG. 6A, cells were stained with ethidium homodimer (red) to label dead cells and with calcein-AM (green) to label live cells. FIG. 6B shows the average ratio of live cells to total cells taken from four images of the two hydrogel blends and 7% methylcellulose, and it shows the results of cell counting by charting the ratio of live cells to total cells. 7% methylcellulose, 7% methylcellulose mixed with 1.5% Seaprep®, and 7% methylcellulose mixed with 1.5% Metaphor showed no significant difference in neuron viability after 48 hours of culture. The results from the NuSieve 3:1® mixture were not included because the hydrogel was not supportive of cell viability experiments. Through repeated experiments, it was discovered that the NuSieve 3:1® hydrogel was quite opaque and fell apart when left in neural growth media for 48 hours, releasing the cells into the media out of the gel. For this reason, the data for this hydrogel was removed from comparison to the other three hydrogels.

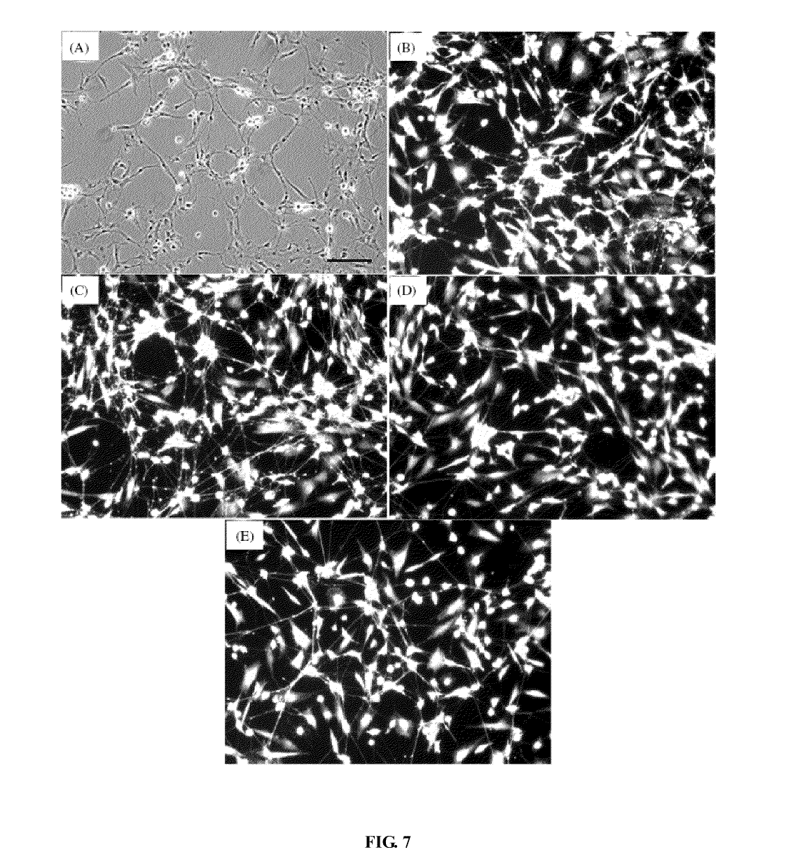

Dissociated DRG cultures were analyzed prior to administration of each of the hydrogels (FIG. 7(A)). Cultures in which 7% methylcellulose (FIG. 7(B)), 7% methylcellulose/1.5% SeaPrep® (FIG. 7(C)), 1.5% MetaPhor®/7% methylcellulose (FIG. 7(D)) or 7% methylcellulose/0.75% NuSieve 3:1® (FIG. 7(E)) were added show that neurons maintained their morphology after addition of the hydrogel. Even though representative images showing dissociated neurons in the presence of hydrogel contained different amounts of cells (due to the uneven distribution of cells on the culture dish), all images showed that even after of administration of hydrogel neurons remained attached to the culture dish and had axons that were connected to each other. This shows that cultures remained viable after the addition of the hydrogel.

### Example 8

### Creation of Agarose/Methylcellulose/Dextran Blend Hydrogels

Materials. The hydrogel being studied was a 7% methylcellulose, 1.5% agarose blend (wt/vol of PBS). The methylcellulose (Dow Chemical Company, Midland, Mich.) used was Methocel A15 Premium LV. Methocel has previously been demonstrated to enhance neural regeneration in the central nervous system (Gupta, D. et al. Biomaterials 2005, 27, 2370-2379). Seaprep® agarose (Lonza, Basel, Switzerland) was chosen because of its ability to foster neural regeneration (Dillon, G. et al. J. Biomater. Sci. Polym. Ed. 1998, 10, 1049-1069). Dextran (Sigma Aldrich, St. Louis, Mo.) was acquired with an average molecular weight of 150 kDa. Methacrylic acid (Sigma-Aldrich, St. Louis, Mo. was obtained with 99% purity. NaOH (Sigma-Aldrich, St. Louis, Mo.) was acquired with an average mass of 0.1 g±0.025 mg per NaOH pellet.

Creation of Methylcellulose/Agarose Blends. The control hydrogel consisted of a 7% methylcellulose, 1.5% agarose hydrogel (wt/vol of PBS) with no additives. The hydrogel was created using 16 mL of phosphate buffered saline (PBS) at physiological pH (7.4). 8 mL PBS was placed in an ice box to remain chilled. The other 8 ml was heated to 80° C. and 225 mg of agarose was added slowly while stirring. Once the solution reached 80° C., 1050 mg of methylcellulose was added and quickly stirred until the methylcellulose was completely wetted (Tate, M. et al. Biomaterials 2001 22, 1113-1123). The chilled PBS was then added and the entire solution was placed in an ice bath and stirred for 10 minutes. The hydrogel was then centrifuged and placed in a refrigerator for future use. In order to characterize the effect of neutral dextran on the hydrogel, its effect on gelation time was examined. For these tests, to the 8 mL of chilled PBS, a known mass of dextran was added and stirred until dissolved. Samples of 20, 40, 60, 80 and 100 mg dextran-containing hydrogels were prepared.

Creation of Cationic Dextran-containing Hydrogel. To determine how the cationic dextran affected the gelation time of the hydrogel, a positive charge had to be induced on the dextran polymer. This was accomplished by first creating a dilute solution of methacrylic acid (MAA). 0.5 mL of MAA (99% purity) was added to 0.5 L of deionized water to create a 12 mM solution (Van Tomme, S. et al. Biomaterials 2005 22, 1197-1203). The volumes of acid were then increased to 1, 1.5, 2 and 2.5 mL of acid added each to 0.5 L purified water to produce 24, 36, 48, and 60 mM solutions, respectively. 40 mg of dextran was then added and stirred until dissolved. The acidic solution was then refrigerated overnight. Once the hydrogel was to be prepared, the acid solution was used to replace 4 mL of the 8 mL chilled PBS used during for hydrogel fabrication. In order to test the effect of varying acid concentration on the gelation time, the mass of dextran added to the hydrogel was chosen to be held constant at 40 mg.

In order to test the effect of varying concentration of dextran on the hydrogel, the concentration of MAA added was held constant at 60 mM. A concentration of 60 mM was chosen to insure that the dextran would become fully protonated for the test masses. The addition of MAA caused the pH to drop, thus, in order to make the hydrogel practical for use in vitro, the pH had to be increased to a physiological range targeted between 7.3 and 7.5. This was accomplished by using sodium hydroxide as the base to neutralize the remaining acid (Nunes, J. et al. Colloids Surf. 2006, 275, 148-152). The amount of sodium hydroxide necessary for neutralization was determined experimentally to be approximately 21 mg. Sodium hydroxide was then added to the dextran-acid solution after it had been allowed to protonate overnight. This resulted in the pH of the dextran-acid solution increasing to within the physiological range (pH 7.3-7.5). The method for creating the hydrogel beyond this point was identical to the control. Since the effect that the sodium hydroxide would elicit on the hydrogel blend was unknown, all tests other than the acid concentration gelation time test and neutral dextran test were performed using both neutralized and non-neutralized samples.

### Example 9

### Gelation Temperature and Time Tests of Agarose/Methylcellulose/Dextran Blend Hydrogels

Gelation Time Tests. To test the gelation time of the hydrogel, 1 mL samples of the hydrogel were pipetted into 1.5 mL microcentrifuge tubes in triplicate (n=3). Three independent hydrogels were created for each group tested. The samples were then blinded by a third party observer. The hydrogel was allowed to equilibrate to room temperature (approximately 20-30 minutes). The tubes were then placed in a warm water bath at 37° C. The tubes were then checked every 30 seconds to see if the gel had solidified. Time was stopped while the gels were out of the water bath and restarted once they were placed back in the water bath. The point of gelation was determined by the inverted test tube method (Dillon, G. et al. J. Biomater. Sci. Polym. Ed. 1998, 10, 1049-1069). It is important to note that the hydrogels were tested within 24 hours after being mixed to ensure that the gels did not undergo any dehydration or other changes in physical properties.

For all other tests except the gelation time tests, five hydrogels were utilized. The first was a control (1050 mg MC, 225 mg agarose), the second and third were samples containing 20 mg and 100 mg dextran with 60 mM MAA that had not been neutralized (referred to as the NN, non-neutralized group), and the fourth and fifth were samples containing 20 mg and 100 mg dextran with 60 mM MAA that had been neutralized to physiological pH using sodium hydroxide (referred to as the NT, neutralized group). A non-neutralized group was tested to ensure that the addition of sodium hydroxide did not reduce the effect of the protonated dextran on reducing the gelation time of the hydrogel.

Gelation Temperature Tests. Gelation temperature was measured using the inverted test tube method (Gupta, D. et al. Biomaterials 2005, 27, 2370-2379). Three test groups were evaluated as previously described: the control, the NN group and the NT group. 1 mL samples of each hydrogel were pipette into 1.5 mL microcentrifuge tubes, blinded by a third party observer, and placed in a hot water bath (n=3). Three independent samples were created for each group tested. The bath was programmed to raise 1° C. per minute from 20° C. to 45° C. The gels were tested every minute until gelation occurred.

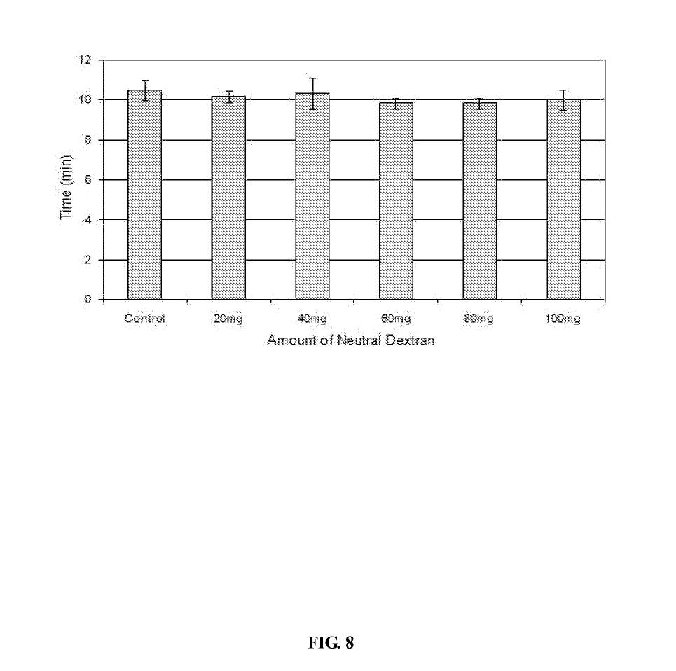

Effect of Neutral Dextran on Hydrogel Gelation Time. FIG. 8 shows the gelation times of hydrogels containing incremental amounts of neutral dextran. Error bars indicate one standard deviation (n=3; each sample independently fabricated). As indicated in FIG. 8, no significant effect on gelation time was elicited by the addition of neutral dextran polymers. Qualitatively, there was also no noticeable change in any of the physical properties of the hydrogel.

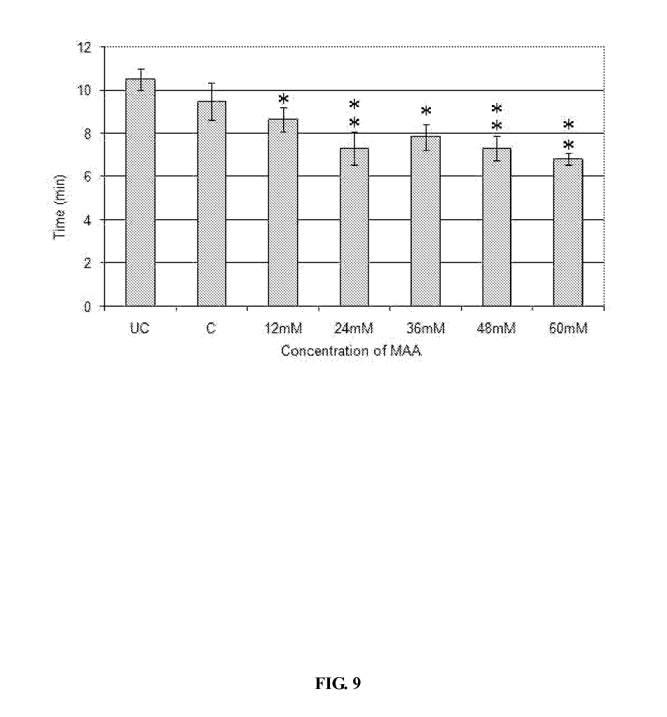

Effect of Acid Concentration on Gelation Time. To test the effect of cationic dextran on the gelation time of the hydrogel, two controls and five test groups were created, each containing 40 mg dextran and 4 mL of MAA. FIG. 9 shows the data obtained from the inverted test tube tests after the addition of positively charged dextran ionized under increasing concentrations of MAA. UC represents the unaltered control, containing 1050 mg MC and 225 mg agarose. C represents a control that was modified to reflect the addition of the 40 mg dextran. This hydrogel had an additional 40 mg MC added to it (1090 mg MC total) to determine if the increase in dry weight was responsible for the decrease in gelation time. There was a modest decline in gelation time, especially when comparing the high concentrations to the control. However, the rate of decline in gelation time decreases with increasing acid concentration. Statistically, all samples were significant when compared to the unaltered control (1050 mg MC, 225 mg agarose). The 24, 48, and 60 mM samples were significant when compared to the altered control. The 36 mM sample did not exhibit significance when compared to the altered control. Statistical analysis was performed using the Tukey-Kramer HSD Test and ANOVA (a=0.05). One asterisk indicates significance when compared to the true control (UC), while two asterisks indicate significance compared to the altered control (C) using the TukeyKramer HSD Test.

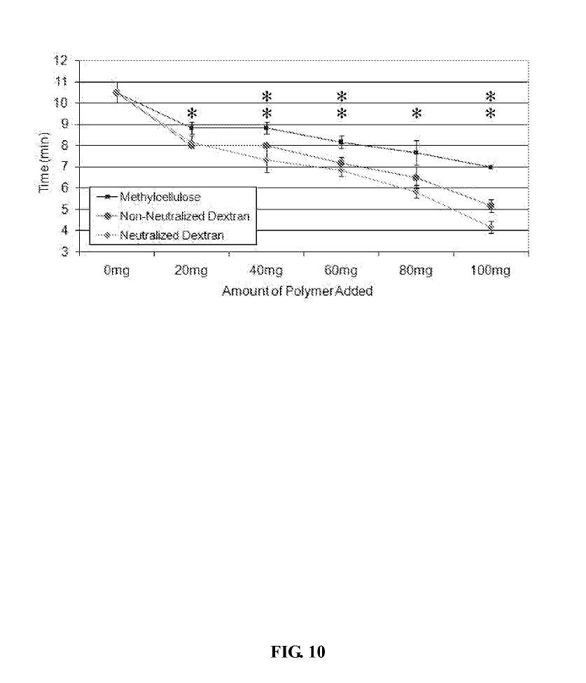

Effect of Increasing Dextran Concentration on Gelation Time. To test the effect of increasing dextran mass on gelation time, the acid concentration was held constant at 60 mM. However, in order to ensure that simply adding more mass (increasing dry weight of the hydrogel) was not responsible for the decrease in gelation time, for each incremental increase in dextran, a control was created containing an identical increase in mass of methylcellulose. All samples were tested in triplicate and compared. FIG. 10 shows the results of the inverted test tube tests. Three lines are plotted indicating the MC control, non-neutralized dextran-containing hydrogels, and a neutralized test group of hydrogels that had been neutralized to physiological pH using NaOH. All samples showed significance when compared to 0 mg control group, and only at 20 mg and 80 mg was there no significance between dextran-containing hydrogels and the methylcellulose-containing hydrogels. The gelation time declines as both methylcellulose and dextran mass are increased. However, the rate of decline in gelation time is one and a half times greater when adding dextran then methylcellulose. Surprisingly, the neutralized hydrogels showed a more rapid gelation than the non-neutralized hydrogels, showing a slope of 0.055 compared to −0.045 for the non-neutralized dextran, which was found by creating a best-fit line for the plot (FIG. 10) and measuring its slope. One asterisk indicates significance when compared to the true control (0 mg MC added), while two asterisks indicates significance compared to the methylcellulose control at that given point using the Tukey-Kramer HSD Test. Since the NT group showed lower gelation times at all points (except for 20 mg dextran) than the NN group, all Tukey-Kramer tests were conducted using the NN group as compared to the control.

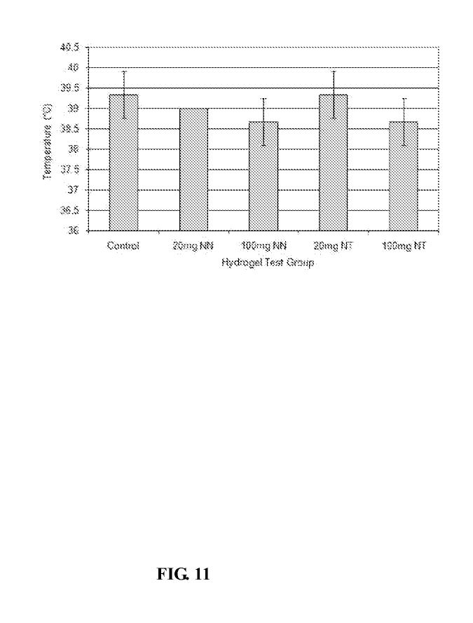

Effect of Dextran on Gelation Temperature. FIG. 11 indicates the effect of dextran on the gelation temperature of the hydrogel. Three groups are represented: a control, non-neutralized dextran-containing hydrogel (NN), and hydrogels that had been neutralized (NT) using NaOH to physiological pH. Error bars indicate one standard deviation (n=3). The dextran did not cause a significant change in the gelation temperature of any of the test groups. All of the groups solidified in approximately 20 minutes as the temperature of the water bath neared 40° C.

### Example 10

### Structural Characterization and Pore Size Evaluation of Agarose/Methylcellulose/Dextran Blend Hydrogels

Pore size is an important determinant of the ability of the material to act as a drug delivery system (Satish, C. et al. Indian J. Pharm. Sci. 2006, 68, 133-140), and pore size plays an important role in determining dorsal root ganglion neurite extension in 3D cultures (Balgude, A. et al. Biomaterials 2001, 22, 1077-1084). The control, NN and NT groups were utilized for this test. Four independent hydrogels were created for each test group, with 4 images taken from each sample (n=16). Each hydrogel sample was pipetted into a mold and allowed to solidify in an incubator at 37° C. for approximately 24 hours. The samples were then removed from their molds and snap frozen using liquid nitrogen and placed in a freeze dryer for 24 hours. The samples were mounted and gold-coated with a thickness of 10 nm using a Hummer 6.2 Sputter Coater (Anatech Ltd, Denver, N.C.). SEM was performed using a JSM 6400 (JEOL Ltd, Tokyo, Japan) scanning electron microscope (SEM) at a magnification of 400× and an accelerating voltage of 10 kV. Four images of each hydrogel sample were taken and analyzed using Carl Zeiss AxioVs40 V 4.6.3.0 imaging software (Carl Zeiss MicroImaging, Inc., Thornwood, N.Y.). Hydrogels were tested in triplicate, for a total of 12 samples of each test group. Pore size was evaluated by measuring the diameter of the pore. For oblong pores, the shortest distance across the pore was taken to be the pore size since it was the limiting diameter.

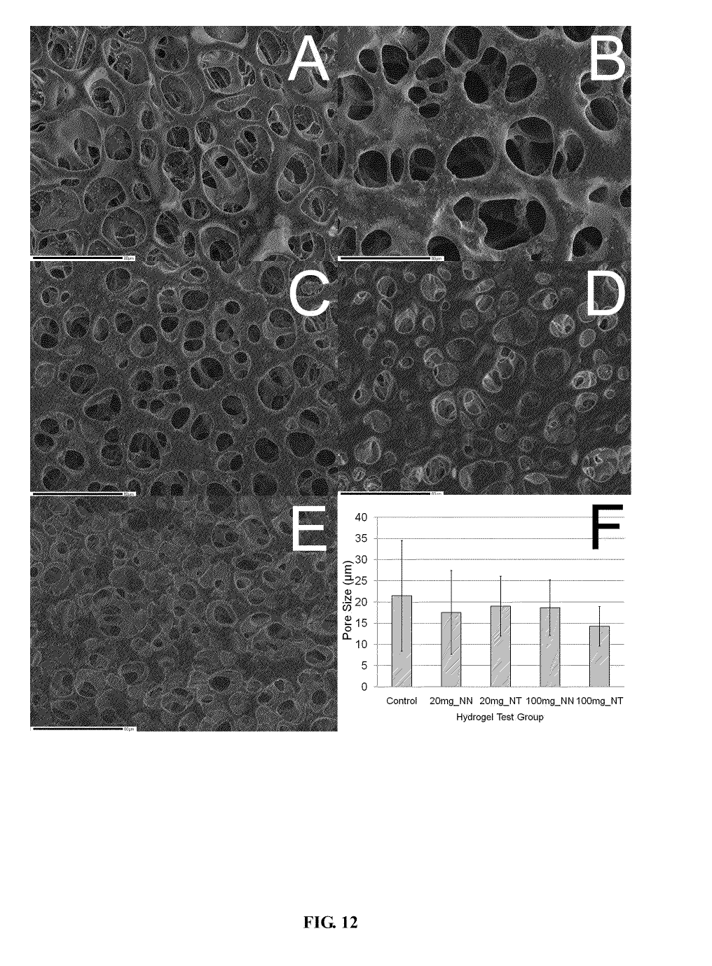

FIG. 12 (A-E) shows five representative images of the pore structure of the test hydrogels: (A) control, (B) 20 mg non-neutralized (NN), (C) 100 mg non-neutralized (NN), (D) 20 mg neutralized (NT), and (E) 100 mg neutralized (NT). All images were taken at 400× magnification with an accelerating voltage of 10 kV. The scale bar represents 80 μm. FIG. 12F shows pore size for each hydrogel group. None of the groups were statistically different from another group. Error bars indicate one standard deviation (n=4). Qualitatively, the control and the 20 mg dextran-containing groups displayed similar overall topographical characteristics. Pore sizes ranged from 1.68 μm to 101.14 μm, with a total average pore size of 18.18 μm. The 100 mg dextran-containing groups appeared to have smaller pore sizes overall, but had more consistency in pore size. These observations are bolstered by the data presented in (F), which displays the average pore size of each group. The control had the largest average pore size. However, the rest of the groups showed no apparent connection between the addition of dextran and pore size. Contrary to this, while the control had a standard deviation in pore size of 13.03, the 100 mg dextran-containing groups (NN and NT) had average standard deviations of 6.58 and 4.66, respectively. This indicates an increase in pore size uniformity as the concentration of dextran increases.

### Example 11

### Rheology of Agarose/Methylcellulose/Dextran Blend Hydrogels

Small amplitude oscillatory rheology was performed utilizing a Bohlin C-VOR Rheometer (Malvern Instruments Ltd, Malvern, Worcestershire UK) to determine the elastic modulus of the hydrogels as a function of time. Frequency sweeps from 10 to 100 radians were performed isothermically at 37° C. for 45 minutes. Each of the hydrogel samples containing various amounts of dextran were heated in a convection oven and maintained at temperature using heated nitrogen gas. Three tests were run for each test group each drawing from the same respective hydrogel (n=3).

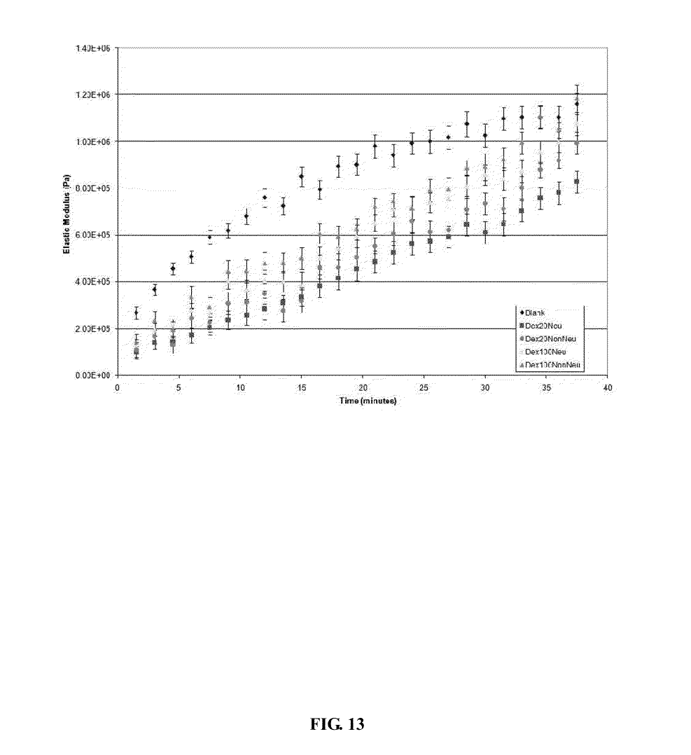

Rheological testing showed that the addition of positively-charged dextran changes the gelation characteristics of the hydrogel. FIG. 13 shows the data gained from the rheological tests. Samples were tested in triplicate (n=3; testing was repeated three times from one fabricated hydrogel) and averaged to yield the results. Error bars indicate on standard deviation. The change in slope of the elastic modulus of the blank control that occurs approximately 10 to 15 minutes into the test identifies that time necessary for gelation to occur. In contrast, all positively-charged dextran hydrogels have no such changes in slope. Statistical analysis (ANOVA, α=0.05) of the results shows that for times between 4.5 and 19.5 minutes, the blank control had a significantly higher elastic modulus than all four dextran-containing hydrogels. For all other times, all five hydrogels showed no significant differences.

### Example 12

### In Vitro Neuronal Response to Dextran-Containing Hydrogels

To determine how dextran-containing hydrogels affected neuronal adhesion and viability, dissociated neurons were cultured in the presence of control hydrogel (no dextran) and neutralized hydrogel containing 100 mg of dextran. To create hydrogel blends for tissue culture, the hydrogel was fabricated in a tissue culture flow hood using autoclaved containers/instruments using previously published protocols (Martin, B. et al. J. Neural Eng. 2008, 5, 221-231). 225 mg of agarose was deposited into 8 mL of 7.4 pH sterile PBS. The contents were heated and stirred until the mixture was clear and colorless. Once colorless, the solution was sterile filtered using a Millex 0.45 μm filter (Millipore, Bedford, Mass.). Methylcellulose powder was sterilized using ethylene oxide gas. The filtered agarose solution was heated to 80° C., and 1050 mg of methylcellulose added. The mixture was then stirred until the methylcellulose was dispersed. The solution was then transferred to an ice bath, and 8 mL of sterile PBS was added. The mixture was then stirred for 10 minutes and then centrifuged to remove air bubbles.

For the dextran-containing hydrogel, 100 mg of dextran was placed into 4 mL of solution containing 60 mM methacrylic acid in deionized water. After protonating overnight, the dextran-acid solution was neutralized using NaOH. This solution was sterile filtered using a Millex 0.22 μm filter (Millipore, Bedford, Mass.). 225 mg of agarose was deposited into 8 mL of 7.4 pH sterile PBS. The contents were heated and stirred until the mixture was clear and colorless. Once colorless, the solution was sterile filtered using a Millex 0.45 μm filter. The filtered agarose solution was heated to 80° C., and 1050 mg of sterile methylcellulose added. The mixture was then stirred until the methylcellulose was dispersed. The solution was then transferred to an ice bath, and 4 mL of sterile PBS and 4 mL of dextran solution was added. The mixture was then stirred for 10 minutes and then centrifuged to remove air bubbles.

Dorsal root ganglia were isolated from embryonic stage nine chick embryos using techniques approved by the Institutional Animal Care and Use Committee at Michigan Technological University. Ganglia were placed into a microcentrifuge tube containing Hank's Balanced Salt Solution (HBSS) (Media Tech, Herndon, Va.) and centrifuged. The HBSS solution was removed and 0.25% trypsin solution was added (Invitrogen, Carlsbad, Calif.). The trypsin solution was allowed to incubate with the ganglia for 10 minutes within a standard tissue culture incubator. The ganglia were then centrifuged, the trypsin solution removed, and growth medium (88% Dulbecco's Modification of Eagle's Medium (DMEM) (Mediatech Inc., Herndon, Va.), 10% fetal bovine serum (FBS) (Hyclone, Logan, Utah) and 2% penicillin/streptomycin (Invitrogen, Carlsbad, Calif.)) added. A fire blown Pasteur pipette was used to dissociate the cells. The suspension was then centrifuged, the growth medium removed, and neural growth medium (Neurobasal medium, supplemented with B-27 non-serum supplement, penicillin/streptomycin, and L-glutamine) (Invitrogen, Carlsbad, Calif.) added. The cells were placed into a small culture dish and allowed to incubate for one hour within a standard tissue culture incubator to facilitate non-neuronal adhesion. After one hour, the culture dish was agitated, and the neural growth medium consisting of neurons was removed and placed into a microcentrifuge tube.

Neurons were plated onto laminin coated 6-well plates (BD Biocoat, San Jose, Calif.) and allowed to attach for 48 hours in a tissue culture incubator in the presence of neural growth medium supplemented with nerve growth factor (NGF) (Invitrogen, Carlsbad, Calif.) so that the final NGF concentration was 50 ng mL-1. After 48 hours, the neural growth medium was removed and approximately 100 μl of chilled hydrogel (refrigerated temperature) was placed on top of the neurons. The cells with hydrogel were placed into a tissue culture incubator for 15 minutes to facilitated hydrogel solidification. To this, neural growth medium supplemented with NGF was again added. The cells were cultured for additional 48 hours and then stained with calcein-AM (Sigma-Aldrich, St. Louis, Mo.).

Neural growth medium was removed from the cultures, and the cultures were washed with PBS. Calcein-AM stock solution (1 mg of calcein-AM in 250 μl of DMSO) was prepared and 5 μl of calcein-AM stock was added to 10 mL of PBS. This solution was added onto the neuronal culture with hydrogel. After 30 minutes, the stain was removed and the culture washed with PBS. The cultures were analyzed and imaged using a Zeiss Axiovert 200M inverted fluorescence microscope with Apotome (Carl Zeiss MicroImaging, Inc., Thornwood, N.Y.). The cultures were analyzed to confirm that the neural cells remained attached to the culture dish (maintaining cellular morphology) without altering cell viability. Each of the hydrogels were tested three times, meaning that the same hydrogel blend was added to cultures prepared during different times (n=3).

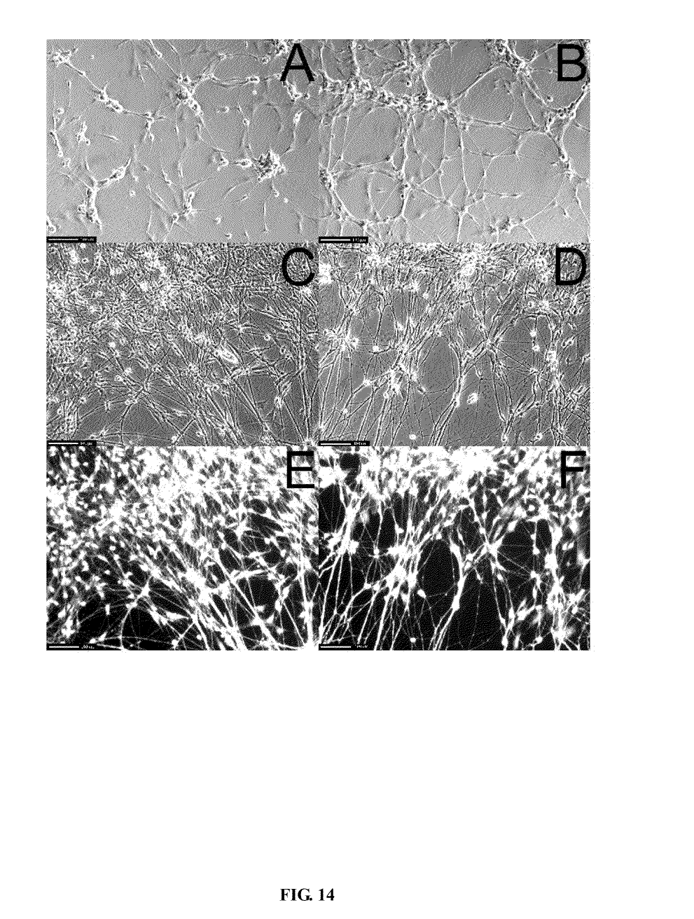

FIG. 14 shows transmitted light images (except 14E and 14F are fluorescent images) of dissociated dorsal root ganglia cells cultured in the presence of an agarose/methylcellulose hydrogel with and without charged dextran. Cultures were repeated three times using one fabricated hydrogel. Cellular studies showed that the addition of a dextran-containing hydrogel onto established dissociated neural cells, followed by subsequent gelation and continued culturing in the presence of the hydrogel did not affect neural adhesion or viability. After 48 hours of culture time and prior to the addition of hydrogel, dissociated dorsal root ganglia cultures were examined and imaged (FIG. 14A—dissociated culture at 48 hours after initial seeding and prior to the additional of the control agarose/methylcellulose hydrogen, i.e., culture what would contain control hydrogel; FIG. 14B—dissociated culture at 48 hours after initial seeding and prior to the additionof agarose/methylcellulose hydrogel containing positively charged dextran, i.e., culture that would contain dextran hydrogel). Control or dextran-containing hydrogel was added to the cultures and allowed to interact with the dissociated neural cultures for 48 hours. Cells were stained with the live cell stain calcein-AM. Transmitted light images of cultures after 48 hours of incubation in the presence of hydrogel showed that neither the control (FIG. 14C, dissociated culture exposed to a plain agarose/methylcellulose hydrogel for 48 hours) nor the dextran-containing hydrogel (FIG. 14D, dissociated culture exposed to dextran-containing agarose/methylcellulose hydrogel for 48 hours) affected neural adhesion. Fluorescent images of the same field of view showed that cultures in the presence of the control hydrogel (FIG. 14E, fluorescent image using a live cell stain of a dissociated culture exposed to a plain agarose/methylcellulose hydrogel for 48 hours) or dextran-containing hydrogel (FIG. 14F, fluorescent image using live cell stain of a dissociated culture exposed to a dextran-containing agarose/methylcellulose hydrogel for 48 hours) remain viable following hydrogel administration.

We claim:

1. A hydrogel comprising:
a) methylcellulose;
b) agarose; and
c) dextran.

2. The hydrogel of claim 1, wherein the dextran is positively charged.

3. The hydrogel of claim 1 comprising about 3% to about 20% (w/v) methylcellulose.

4. The hydrogen of claim 3 comprising about 5% to about 9% (w/v) methylcellulose.

5. The hydrogel of claim 3 comprising about 7% (w/v) methylcellulose.

6. The hydrogel of claim 3 comprising about 10% to about 12% (w/v) methylcellulose.

7. The hydrogel of claim 1 comprising about 0.5% to about 5% (w/v) agarose.

8. The hydrogel of claim 7 comprising about 1.5% (w/v) agarose.

9. The hydrogel of claim 1 comprising about 20 to about 500 mg of dextran per about 15 mL hydrogel.

10. The hydrogel of claim 10 comprising about 100 mg of dextran per about 15 mL hydrogel.

11. The hydrogel of claim 1, further comprising at least one additional bioactive component.

12. The hydrogel of claim 11, wherein the at least one additional bioactive component is selected from the group consisting of cells, peptides, polypeptides, polymers and small molecules.

13. The hydrogel of claim 12, wherein the at least one additional bioactive component is selected from the group consisting of platelets, Schwann cells, neurons, keratinocytes, fibroblasts, chondrocytes, bone formation cells, myocytes and stem cells.

14. The hydrogel of claim 12, wherein the at least one additional bioactive component is selected from the group consisting of cytokines, glutathione, parathyroid hormone, growth factors, neutrotrophic factors, coagulation agents, laminin, fibronectin, collagen and proteoglycans.

15. The hydrogel of claim 12, wherein the at least one additional bioactive component is polyethylene glycol.

16. The hydrogel of claim 12, wherein the at least one additional bioactive component is selected from the group consisting of antibiotics, anti-fungals, coagulation agents and immunosuppressants.

17. A composition comprising the hydrogel of claim 1 and a scaffold.

18. The composition of claim 17, wherein the scaffold comprises calcium phosphate.

19. The composition of claim 17, wherein the scaffold is a nerve guidance channel.

20. The composition of claim 19, wherein the nerve guidance channel comprises a set of aligned polymer fibers.

21. The composition of claim 20, wherein the polymer fibers comprise at least one polymer selected from the group consisting of poly-L-glycolic acid, poly-L-lactic acid, blends of poly-L-glycolic acid and poly-L-lactic acid, collagen, laminin, fibrin and combinations thereof.

22. A method of making a hydrogel according to claim 1 comprising:
a) combining a suspension of agarose and methylcellulose and a dextran solution; and
b) mixing the suspension and solution at low temperature.

23. The method of claim 22, wherein at least one additional bioactive component is added.

24. A method of treating nervous system injury comprising administering the hydrogel according to claim 1 to a subject in need thereof.

25. The method of claim 24, wherein the nervous system is the peripheral nervous system.

26. The method of claim 25, wherein the central nervous system injury comprises a spinal cord injury.

27. The method of claim 24, wherein the nervous system is the peripheral nervous system.

28. The method of claim 27, wherein the peripheral nervous system injury is diabetic neuropathy.

29. A method of regenerating tissue comprising contacting damaged tissue with the hydrogel according to claim 1.

30. The method of claim 29, wherein the tissue is selected from the group consisting of nerve tissue, connective tissue, muscle tissue and skin.

31. A method of enhancing tissue regeneration comprising administering the hydrogel according to claim 1 to a subject in need thereof.

32. The method of claim 31, wherein the tissue is selected from the group consisting of nerve tissue, connective tissue, muscle tissue and skin.

33. A method of treating a subject having an artificial joint comprising administering the hydrogel according to claim 1 to the artificial joint site.

34. A method of facilitating nerve growth comprising contacting a region in need of nerve growth with a hydrogel according to claim 1.
